SEQUENCE LISTING (21 sequences)

SEQ ID NO: 1 - DNA, 78 bp, Artificial Sequence
  Oligonucleotide
ttgaaagatctgaattcatgcaccaccatcaccatcacggatccggcggcggcggctcggcggctagtgcccgtgggt

SEQ ID NO: 2 - DNA, 71 bp, Artificial Sequence
  Oligonucleotide
ttctgggtaccgctttaatggtgatgatggtggtgtctagagctttaactagtgacggctattcctccacc

SEQ ID NO: 3 - DNA, 42 bp, Artificial Sequence
  Oligonucleotide
cgggatgaattcgtcgccaccatggtgagcaagggcgaggag

SEQ ID NO: 4 - DNA, 33 bp, Artificial Sequence
  Oligonucleotide
gcggccggatcccttgtacagctcgtccatgcc

SEQ ID NO: 5 - DNA, 33 bp, Artificial Sequence
  Oligonucleotide
gtcgccactagtgtgagcaagggcgaggagctg

SEQ ID NO: 6 - DNA, 39 bp, Artificial Sequence
  Oligonucleotide
agagtcactagtgctttacttgtacagctcgtccatgcc

SEQ ID NO: 7 - DNA, 36 bp, Artificial Sequence
  Oligonucleotide
gttattcatgagatctgtcaatgccaataggatatc

SEQ ID NO: 8 - DNA, 31 bp, Artificial Sequence
  Oligonucleotide
ttaggtcatgaacatatacctgccgttcact

SEQ ID NO: 9 - DNA, 54 bp, Artificial Sequence
  Oligonucleotide
aaatatgaggagttacaattgctaattaattaattcggggaaatgtgcgcggaa

SEQ ID NO: 10 - DNA, 33 bp, Artificial Sequence
  Oligonucleotide
cttggtcctaggattaccaatgcttaatcagtg

SEQ ID NO: 11 - DNA, 85 bp, Artificial Sequence
  Oligonucleotide
caaataatgattttattttgactgatagtgacctgttcgttgcaacaaattgataagcaatgcttttttataatgccaactttgt

SEQ ID NO: 12 - DNA, 80 bp, Artificial Sequence
  Oligonucleotide
caaataatgattttattttgactgatagtgacctgttcgttgcaacaaattgataagcaatgctttcttataatgccaac

SEQ ID NO: 13 - DNA, 81 bp, Artificial Sequence
  Oligonucleotide
cgctctggcgcttgccttcgccgccgttacggccgcgggtgttgccgcggctcagaccgtggccagaaagtgctcgctgac

SEQ ID NO: 14 - DNA, 79 bp, Artificial Sequence
  Oligonucleotide
gcttttttataatgccaactttgtacaaaaaagcaggctatgaacaaaccctccaaattcgcgctggcgcttgccttcg

SEQ ID NO: 15 - DNA, 63 bp, Artificial Sequence
  Oligonucleotide
tgctttcttctaatgccaactttgtacaagaaagctgggtattactccttctgtgtgcgcagg

SEQ ID NO: 16 - DNA, 51 bp, Artificial Sequence
  Oligonucleotide
ttataatgccaactttgtacaaaaaagcaggctatggtgagcaagggcgag

SEQ ID NO: 17 - DNA, 58 bp, Artificial Sequence
  Oligonucleotide
tgctttcttataatgccaactttgtacaagaaagctgggtttacttgtacagctcgtc

SEQ ID NO: 18 - DNA, 44 bp, Artificial Sequence
  Forward primer
gctgtggatccggcggcggcggctcgaacacggacgctcagtcg

SEQ ID NO: 19 - DNA, 32 bp, Artificial Sequence
  Reverse primer
ccttaactagttttattcaggcacattaaatc

SEQ ID NO: 20 - DNA, 29 bp, Artificial Sequence
  Forward primer
aaggaactagtgtgagcaagggcgaggag

SEQ ID NO: 21 - DNA, 29 bp, Artificial Sequence
  Reverse primer
tcgaaactagtcttgtacagctcgtccat

(19) United States
(12) Patent Application Publication
(10) Pub. No.: US 2009/0176660 A1
(43) Pub. Date: Jul. 9, 2009

(54) Engineered Baculoviruses and Their Use

(76) Inventors: Seppo Yla-Herttuala, Kuopio (FI); Kari Juhani Airenne, Kuopio (FI)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/327,423

(22) Filed: Dec. 3, 2008

Related U.S. Application Data
(63) Continuation-in-part of application No. 10/507,268, filed on Sep. 9, 2004, now abandoned.

(30) Foreign Application Priority Data
Mar. 12, 2002 (GB) PCT/GB02/01115

(51) Int. Cl. C40B 30/06; C12N 7/01; C12N 5/00; C12N 5/06

(52) U.S. Cl. 506/10; 435/235.1; 435/325; 435/348

(57) ABSTRACT

Baculovirus is engineered so that the capsid displays one or more heterologous peptides or protein. Such baculovirus can be used to deliver therapeutics, and in functional genomics.

Also a method for generating recombinant baculoviruses comprises:
(i) incorporating a lethal gene into a donor plasmid comprising an expression cassette;
(ii) transposing the expression cassette from the donor plasmid into a bacmid in E. coli cells to form a recombinant bacmid, wherein the lethal gene product kills the cells still harbouring the donor vector;
(iii) extracting the recombinant bacmids; and
(v) transfecting insect cells with recombinant bacmids to form recombinant baculoviruses.

### CROSS-REFERENCE TO A RELATED APPLICATION

This application is a continuation-in-part application of co-pending Application U.S. Ser. No. 10/507,268, filed Sep. 9, 2004, which is a National Stage Application of International Application Number PCT/GB03101029, filed Mar. 12, 2003; which claims priority to International Application Number PCT/GB02/01115, filed Mar. 12, 2002; all of which are incorporated herein in their entireties.

### FIELD OF THE INVENTION

This invention relates to engineered baculoviruses and their use, and especially to libraries and peptide display provided in baculovirus.

### BACKGROUND OF THE INVENTION

Over the past few years, many organisms have had their genomes completely sequenced. A draft sequence of the entire human genome has been published. However, sequence information as such does not explain what all the genes do, how cells work, how cells form organisms, what goes wrong in disease, how we age or how to develop a drug. This is where functional genomics, an area of the post-genomic era that deals with the functional analysis of genes and their products, comes into play.

Among the techniques of functional genomics, both DNA microarrays and proteomics hold great promise for the study of complex biological systems. Although DNA microarrays allow high throughput analysis of transcriptome (the complement of mRNAs transcribed from a cell's genome at any one time), genes may be present, they may be mutated, but they are not necessarily transcribed. Some messengers are transcribed but not translated, and the number of mRNA copies does not necessarily reflect the number of functional protein molecules. Proteomics (the complete set of proteins encoded by a cell at any one time) addresses problems that cannot be approached by DNA analysis, namely, relative abundance of the protein product, post-translational modification, subcellular localisation, turnover, interaction with other proteins as well as functional aspects.

The observable characteristics conferred by a gene in an expression library allow the discovery of functional open reading frames in new sequenced genomes (genomic library) as well as the characterisation of function of unknown genes (genomic or cDNA library). A library compatible at the same time with bacterial and eukaryotic cells as well as with in vitro and in vivo experiments would be a powerful tool in this sense. Although a plasmid vector could allow this in theory, the inefficiency of transduction of eukaryotic cells by plasmid DNA, not to mention the modest gene transfer efficiency of plasmids in vivo, decreases the usefulness of plasmid libraries as high throughput tools of phenomics (automated/high throughput analysis of proteins).

Baculoviruses have long been used as biopesticides and as tools for efficient recombinant protein production in insect cells. They are generally regarded as safe, due to their naturally high species-specificity and because they are not known to propagate in any non-invertebrate host. Baculoviruses are large enveloped insect viruses. The cigar-shaped nucleocapsid of the baculovirus encloses a 134 kb sized DNA genome. Baculoviruses exist in two forms during natural infection. An occlusion derived virus, ODV, transmits infection from host to host and a budded virus, BV, spreads the infection within the host. The baculovirus Autographa californica multicapsid nuclepolyhedrovirus (AcMNPV) has many features that make it a promising new tool for gene therapy. Baculoviruses have very restricted host range and they do not replicate in vertebrate cells, yet AcMNPV, having an eukaryotic promoter, can effectively transduce mammalian cells. Baculovirus -derived vectors can carry over 50 kb of foreign DNA in their genome, which enables a delivery of complex constructs into target cells.

The Autographa californica multiple nuclear polyhedrosis virus (AcMNPV), containing an appropriate eukaryotic promoter, is able to efficiently transfer and express target genes in several mammalian cell types in vitro. Further, as reported in WO-A-01/90390, baculoviruses are able to mediate in vivo gene transfer comparable to adenoviruses; see also Airenne et al, Gene Ther. 7:1499-1504 (2000). The ease of manipulation and rapid construction of recombinant baculoviruses, the lack of cytotoxicity in mammalian cells, even at a high multiplicity of infection, an inherent incapability to replicate in mammalian cells, and a large capacity (no known insert limit) for the insertion of foreign sequences, are features of baculovirus.

Although the 3-D structure for some AcMNPV proteins is known, virion structure of the AcMNPV remains to be resolved. This is mostly because the virion is large and complex. However, the virus genome was sequenced in 1994 (Virology. 1994 Aug. 1; 202(2):586-605.). This allowed first prediction and later experimental verification (function and possible location in the virion) of the AcMNPV open reading frames. Phylogenetic studies, with over 29 sequenced baculovirus genomes, have also provided further information of the BV proteome. The recent and comprehensive review by Slack and Arif (incorporated herein by reference in its entirety) summarises the current knowledge of the virion structure and function (Adv Virus Res. 2007; 69:99-165).

The nucleocapsids of the ODV and BV have many similarities as they both contain complete viral genomes and have major proteins in common. The most abundant structural protein of the nucleocapsid is vp39. In addition to the major VP39 capsid protein, there are a number of other minor, but important, capsid-associated proteins. The PP78/83 (ORF1639) protein is a phosphoprotein that was first identified in viral fractionation studies as BV/ODV envelope protein and/or ODV tegument protein. EM studies revealed that PP78/83 is associated with the nucleocapsid base. Discovery that PP78/83 nucleates actin polymerisation has lead to the suggestion that this protein is involved in nucleocapsid translocation into the nucleus after infection.

BV/ODV-C42 is a capsid-associated protein that interacts directly with PP78/83. BV/ODV-C42 has a conserved nuclear localisation signal and localises along with PP78/83 in the DNA-rich virogenic stroma. BV/ODV-C42 has not been specifically localised to the capsid base; it interacts directly with another highly conserved capsid-associated viral protein called ODV-EC27.

The vp80 protein is a capsid-associated structural protein that was first identified as P87 in OpMNPV. Gene homologues to the vp80 gene are only found in NPV genomes. The vp80 gene is transcribed late in infection and the protein localizes in the nucleocapsids of BV and ODV. The vp80 homologue of CfMNPV has 72 and 82 kDa molecular weight protein forms and only the 82-kDa protein is associated with ODV nucleocapsids. P24 is another capsid associated protein.

Vp39 is a major capsid protein of baculovirus. Baculovirus enters the cells via receptor-mediated endocytosis. The virus is efficiently internalised by many mammalian cell lines, but is not able to enter the nucleus in non-permissive cells.

It has been previously suggested that the block of an efficient transduction of mammalian cells is not the lack of penetration of the baculovirus into the cells by endocytosis, but the incapability of the virus to reach the nucleus (Boyce, PNAS USA 93:2348-2352, 1996; Barsoum, Hum. Gene Ther. 8:2011-2018, 1997). There is a general assumption that the block of transduction is in the virus escape from the endosomes.

It is known to engineer the major surface glycoprotein of AcNPV, for the presentation of heterologous proteins on the virus surface (Boublik et al., Biotechnology (N.Y.) 13: 1079-1084, 1995). Reference may also be made to O'Reilly et al, “Baculovirus expression vectors. A laboratory manual”, Oxford University Press, New York, N.Y. (1994).

In order to avoid laborious and time-consuming plaque purification processes, genetic material can be introduced into the baculovirus genome by homologous recombination in the yeast Saccharomyces cerevisae ; see Patel et al. Nucleic Acids Res. 20, 97-104, 1992. This method is rapid (pure recombinant virus within 10-12 days) and it ensures that there is no parental virus background but suffers from the need for experience in yeast culturing and the incompatibility of traditional transfer vectors with the system.

Luckow et al., J. Virol. 67, 4566-4579, 1993, describes a faster approach (pure recombinant virus within 7-10 days) for generation of recombinant baculoviruses, which uses site-specific transposition with Tn7 to insert foreign genes into bacmid DNA (virus genome) propagated in E. coli cells. The E. coli clones containing recombinant bacmids are selected by colour (β-galactosidase), and the DNA purified from a single white colony is used to transfect insect cells. This system is compatible for simultaneous isolation of multiple recombinant viruses but suffers from the relative low percentage of recombinant colonies (baculovirus genomes) obtained upon transformation.

The poor selection features of the original system have been enhanced by a temperature-sensitive selection procedure, as described by Leusch et al, Gene 160, 191-194, 1995. However, this system has proved to be uncertain in use.

### SUMMARY OF THE INVENTION

According to a first aspect of the present invention, a method for selecting a target gene, comprises the steps of:

(i) generating a library of genes or genomic fragments cloned in baculovirus as a vector;

(ii) transforming a host cell with the vector; and

(iii) detecting gene expression under predetermined conditions.

Baculoviral genomic or cDNA libraries offer a powerful tool for phenomics, by enabling the functional screening of the constructed libraries in eukaryotic cells both in vitro and in vivo. Addition of a bacterial promoter into a baculovirus donor vector will also allow expression screening of cDNA libraries in bacterial cells. Baculovirus libraries may be constructed from suitable validated full-length clones and sequences from human and other vertebrate sources. This will allow integration of the efficient infection (insect cells) and transduction (vertebrate cells) of target cells by baculoviruses, and application to phenomics.

According to a second aspect of the invention, the baculovirus capsid is modified to display one or more heterologous proteins or peptides (the latter term is used generally herein, to include proteins). Baculovirus correspondingly modified in its genome represents a further aspect of the invention. Such baculovirus can be used to transduce mammalian and other cells. In particular, it has now been shown that the major block in baculovirus transduction of mammalian cells is not in endosome escape, but in nuclear transport of the virus capsid.

It has also been shown herein that, in particular embodiments of the invention, new protein entities can be fused to the N- or C-terminus of vp39 or p24, without compromising the viral titer and functionality of the vp39 or p24 fusion proteins on the AcMNPV capsid surface. Furthermore, the tagged virus can be used for gene transfer in vivo. The constructed baculovirus thus provides a versatile tool for real-time analysis of the transduction route of AcMNPV in mammalian cells and intact animals as well as infection mechanism in insect cells. Capsid-modified baculoviruses also hold a great promise for the nuclear and subcellular targeting of transgenes and as a new peptide display system for eukaryotic cells.

Vp39 and p24 are used to illustrate the invention only. It will be apparent to those skilled in the art that the present invention is applicable to all capsid proteins. By utilising the techniques disclosed herein for forming vp39 and p24 capsid display systems, the skilled person can apply the teaching to all capsid proteins.

Slack and Arif (Adv Virus Res. 2007; 69:99-165), which is incorporated herein by reference in its entirety, have summarised the current knowledge of the virion structure and function. Based in this information, the skilled person can construct desired fusion proteins based on any capsid protein. It is well-known to those skilled in the art that sequence information is easily retrieved form databanks, such as the National Centre for Biotechnology Information (NCBI).

The capsid display system has many advantages compared to a gp64 envelope display system. In vp39, for example, no structural motifs have been recognised either for association with molecules within the stromal matter or for capsid assembly, nor is it responsible for infectivity of the virus. In addition, immunoelectron microscopy shows that vp39 is randomly distributed on the surface of the capsid as opposed to gp64 on the virus envelope. Baculovirus envelope display system allows only fusions to N-terminal end of the gp64, whereas vp39 allows tagging to both terminus. Although it remains to be shown how large proteins can be, displayed on the baculovirus capsid, results suggest that at least 27 kDa protein can be efficiently expressed. Because the length of the capsid can extend relatively freely, it is reasonable to expect that vp39 is also compatible with larger proteins, e.g. up to 100 kDa or higher. Random display of peptides or proteins on the capsid may allow the discovery of moieties capable of transporting the capsid into the nucleus or other intracellular organels.

This invention also provides an improved method for the generation of recombinant baculoviruses by Tn7-mediated transposition. The method is based on a modified donor vector and an improved selection scheme of the baculovirus bacmids in E. coli with SacB gene. Recombinant bacmids can be generated at a frequency of ≧10⁵ per μg of donor vector with a negligible background. This easy-to-use and efficient system provides the basis for a high-throughput generation of recombinant baculoviruses as well as a more convenient way to produce single viruses. The introduced selection scheme may also be useful for the construction of other vectors by transposition in E. coli.

Further uses for modified baculovirus according to the invention include any form of “capsid therapy”. Thus, proteins can be used as a system for the transport of peptides or proteins directly into the nucleus.

In particular, the concept of baculovirus-mediated therapy includes the possibility of using baculovirus capsid as a shuttle for the transport of therapeutic proteins into cells as an alternative to traditional protein transduction schemes. The benefits of therapy without a need for transgene expression are evident.

The baculovirus capsid display system offers a facile tool to study baculovirus transduction mechanisms in the mammalian cells as well as infection mechanisms in the insect cells. In addition, this system provides a novel tool both to the expansion of the baculovirus targeting possibilities at intracellular level and to enhance the display of complex peptides and proteins. Furthermore, the EGFP baculovirus construct provides a valuable tool to study real time entry and intracellular movement of the virus in mammalian cells as well as tracking biodistribution and transduction in vivo.

A further aspect of the invention is a novel tetra-promoter vector (pBVboostFG) that enables screening of large insert-containing libraries in bacterial, insect and mammalian cells. Cloning of the desired DNA fragments is based on the efficient site-specific recombination system of bacteriophage lambda. In addition, the vector is compatible with the improved mini Tn7-based transpositional cloning system, pBVboost, that enables easy and fast production of recombinant baculoviruses without any background. The vector contains the following promoters: chicken β-actin, T7lac, p10 and pPolh, which can be used to express the cloned inserts in mammalian, bacterial and insect cells. By means of the invention, the test genes chicken avidin and enhanced green fluorescent protein (EGFP) were cloned easily and effectively into the new vector and expressed in host cells. By using this vector, it is possible to screen large libraries, in the scale of whole genomes, thus making pBVboostFG a tool for functional genomics.

The cloning of the libraries to the developed vector is based on the efficient site-specific recombination system of bacteriophage lambda. The cloned libraries can be easily transferred to any other system, based on the same recombinational cloning schema. In addition, transduction of the cloned genes can also be done directly in vivo without any further subcloning steps, via baculovirus-mediated transduction. In contrast to adenovirus and retrovirus-based systems, a benefit obtained by using baculovirus as a library-containing vector is that there is no known upper limit of the insertional DNA that can be incorporated in its genome.

### BRIEF DESCRIPTION OF THE DRAWINGS

FIG. 1 is a map of the capsid display plasmid pBACcap-1. The plasmid is designed for baculovirus capsid display by N-terminal or C-terminal fusion of peptides or proteins with the AcMNPV capsid protein vp39.

FIG. 2 is a plasmid map of pBVboost donor vector. The insect cell expression cassette is composed of a multiple cloning site (MCS, unique restriction enzymes shown) flanked by the polyhedrin promoter (ppolh) and simian virus 40 polyadenylation site (SV40 pA). Tn7L and Tn7R, left and right ends of the Tn7 cassette; SacB#3, mutated levansucrase gene; ori, the ColE1 origin of replication; GENT, gentamycin gene.

FIG. 3 is a map of the pBVBoostFG vector. The vector is designed for efficient construction of baculovirus expression libraries by RC system of bacteriophage lambda but includes also an option for traditional restriction enzyme-based library construction. The system allows expression of desired genes under a universal (hybrid tetra-promoter) system which enables simultaneous characterization of the activity of the cloned open reading frames in E. coli as plasmid library or as baculoviral library in insect and mammalian cells and animals. Cloning of the marker gene under pPolh promoter can be used for easy detection of produced baculoviruses as in the case of pBVboostFGR or to modify the produced baculoviral library by other means.

FIG. 4 is an overview of the use of pBVboostFG-based system to clone and generate universal baculoviral libraries. The steps that are shown are as follows:
1. RC clone desired library into RC casette of pBVboostFG.
2. Transform E. coli DH10BacΔTn7 cells with recombinant pBVboostFG library.
3. Gentamycin, tetracycline and sucrose selection results in 100% recombinant bacmids.
4. Transfer colonies and grow overnight.
5. Extract recombinant bacmids by alkaline lysis and transfect insect cells.
6. Primary virus screening. Titer ˜10⁷ pfu/ml.
7. Transduce in desired target cells and test in vivo.

FIG. 5 is a schematic of the SES-PCR strategy to construct avidin (A) and EGFP (B) cassettes for cloning into pBVboostFG. The undermost dashed lines show the attL sites compatible with LR reaction of the used RC system and bacterial ompA signal (in avidin) in oligonucleotides. (C) Oligonucleotides to synthesize avidin and EGFP constructs compatible with LR reaction. attL-sequences are shown in italics and a sequence encoding omp A signal peptide is underlined.

FIG. 6 is a map of p24 and vp39 capsid display plasmids.

### DESCRIPTION OF PREFERRED EMBODIMENTS

In order to direct a high level expression of baculovirus library genes in invertebrate, E. coli, and insect cells, an expression cassette may be constructed, based on a hybrid or other suitable promoter which allows high level expression of target genes both in prokaryotic and eukaryotic cells. A target site for, say, cre-recombinase (loxP) may be included into the expression cassette, to allow easy construction of baculovirus libraries using site-specific recombination in vitro (Sauer, Methods 14:381-392, 1998). To further increase the options to construct the baculovirus libraries, attR and ccdB sites (and, say, a chloramphenicol-resistance or other marker to select for successful ligation of the cassette) can be included into expression cassette. This enables facile conversion of libraries, compatible with, say, Life Technologies Gibco BRL® Gateway™ Cloning Technology (Life Technologies), to the novel baculovirus library. In addition to cre/lox and Gateway compatibility, the expression cassette can allow traditional library construction by several unique restriction enzymes available in vector MCS after modifications such as those described above.

The constructed expression cassette may be cloned into any suitable baculovirus plasmid or baculovirus system which can act as a donor vector. pFastBac-1 is a preferred backbone plasmid since it is compatible with Bac-To-Bac™ baculovirus expression system (Gibco BRL) which allows rapid and easy preparation of re-baculoviruses by site-specific transposition in Escherichia coli. If desired, the cassette can also be integrated to any desired plasmid/expression system, e.g. into a version of Bac-TO-Bac™ baculovirus expression system that permits more efficient and direct construction of baculoviruses (Leusch et al, Gene 160:191-194, 1995).

The expression cassette can also be cloned as part of the baculovirus genome and library construction then performed directly to it by cre/lox, Gateway or direct cloning methods.

All cloning work can be performed using standard molecular biology methods. Constructed baculovirus libraries will be screened for expression/phenotype effect(s) in suitable E. coli strain(s) (library in donor plasmid format), insect cells and vertebrate cells. Selected viruses or whole libraries can also be used directly for in vivo studies. This alleviates the great and unique potential of the new baculovirus libraries; the same library can be used for prokaryotic and eukaryotic cells and in cell (in vitro) and animal (in vivo) studies.

By way of example, and in order to allow intracellular targeting of AcMNPV, a baculovirus capsid display system has been developed. The system is based on a versatile donor vector which allows efficient production of desired proteins as N- or C-terminal fusion to the baculovirus major capsid protein, vp39 (Thiem & Miller, J. Virol. 63:2008-2018, 1989). Alternative baculovirus capsid proteins which are potential targets for peptides or proteins include p24 and p80.

A construct of high titer re-AcMNPV can display a high concentration of a foreign protein in its capsid. The tagged virus is a facile tool to study the route of baculovirus transduction in mammalian cells from the cell surface into the nucleus and transfection capacity of baculovirus in vivo. The system provides at the same time a powerful tool to study the bottlenecks of AcMNPV transduction of non-permissible cell lines and a possibility to improve nuclear or subcellular targeting by incorporation of specific sequences in vp39 protein. AcMNPV may also allow double-targeting at the cell surface level by insertion of specific ligands or antibodies to the envelope, followed by intracellular targeting by vp39 modification.

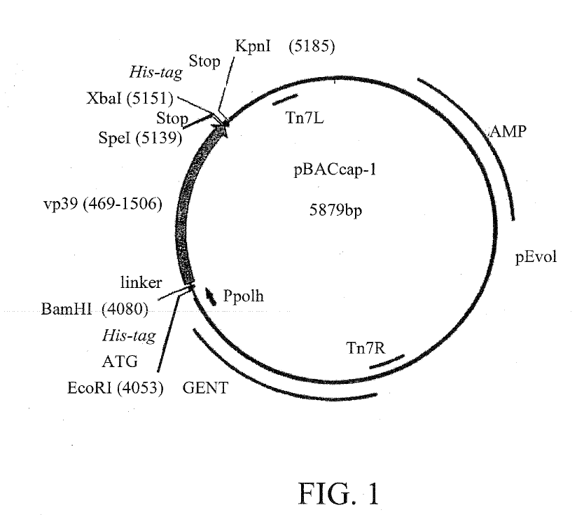

To maximise the chance to achieve a functional fusion and capsid assembly, a transfer plasmid was constructed which enables fusion of desired entities either into N- or C-terminus of the vp39 (FIG. 1). Fusion protein production is driven by a strong polyhedrin promoter, e.g. as disclosed by O'Reilly et al, supra. Since computer prediction showed that vp39 had low complexity at C-terminus but was constrained at N-terminus, a linker sequence (e.g. GGGGS) may be added to the N-terminus, to give distance and flexibility for N-terminal fusion proteins to fold correctly. An option to tag the vp39 fusion proteins with a His-tag may also be preferred. For example, the pBACcap-1 plasmid produces vp39 with His-tag at the N-terminus. However, the same transfer plasmid can be used for N- or C-terminal fusions with or without His-tag. The system is compatible with transposon-mediated virus preparation. However, the expression cassette in the pBACcap-1 can be easily moved to any desired baculovirus vector.

The present invention includes the possibility of double-targeting, as an extension of the conventional targeting working primarily at tissue or cell surface level. The basic idea of the tissue targeting is to add a specific ligand on the surface of the gene transfer vector to achieve specific binding to desired cells or tissues. It is well known that a specific ligand-receptor interaction does not guarantee efficient transduction of the target cell. Internalisation, escape from endosomes and transport of the genetic material into nucleus are also required. Although the transduction can be improved by selection of cell membrane targeting moieties, the route from cytosol to nucleus remains difficult to achieve. Enveloped viruses hold a promise for an efficient double-targeting at the tissue and intracellular levels. By modifying the envelope with a desired tissue targeting moiety and the capsid with an intracellular targeting moiety, efficient and specific transduction of the target cells should be achieved. Transcriptional targeting with specific promoters may also be added to these vectors.

A method of the invention, for the improved generation of recombinant baculoviruses, involves incorporating a lethal gene into the donor plasmid. The lethal gene product may kill cells still harboring the donor vector while the combined selection pressure as a result of the successful transposition of the expression cassette from the donor plasmid into the bacmid may effectively rescue only recombinant-bacmids. In a particular embodiment, a donor vector pBVboost carries the SacB gene from Bacillus amyloliquefaciens; see Tang et al., Gene 96, 89-93, 1990. SacB encodes levansucrase which catalyses the hydrolysis of sucrose to generate the lethal product levan. Levan will kill cells in the presence of sucrose. It may be effective to use a mutated gene, in order to balance the lethal effect of levan in the presence of sucrose with the additional antibiotic pressure.

It appears that cloning of a transgene into pBVboost does not affect the improved selection scheme. The yields and expression characteristics of these viruses are generally similar or identical to viruses generated by other systems. High-titer viruses are generated, capable of expressing large quantities of desired gene products in insect cells or, with a suitable promoter, in mammalian cells; see Airenne et al (2000), supra. However, a striking difference as compared to the original method is that bacmid recombinants can be generated at a frequency of ≧10⁵ per μg of donor vector with a negligible background. This frequency may further be improved by optimising the preparation of competent DH10BacΔTn7 cells and by further optimising the transformation protocol. An additional advantage of the pBVboost system is that due to the powerful selection scheme there is no need for colour selection (i.e. no need for expensive X-Gal and IPTG in the plates). This makes the system cost-effective.

In conclusion, the use of the presented new selection scheme by-passes the disadvantages associated with the original transposition-based generation of baculovirus genomes in E. coli while retaining the simple, rapid and convenient virus production. Addition of the lethal gene into the donor plasmid along with an E. coli strain, in which the chromosomal attTn7 is occupied, permits efficient selection of the recombinant bacmids in a cost-effective manner. The improved pBVboost system is compatible with high-throughput applications like expression library screening but enhances also the construction of single recombinant viruses.

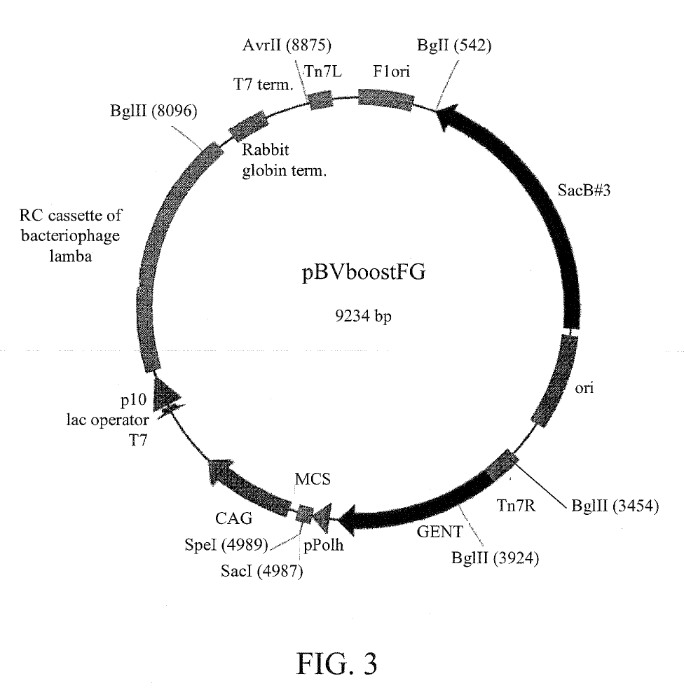

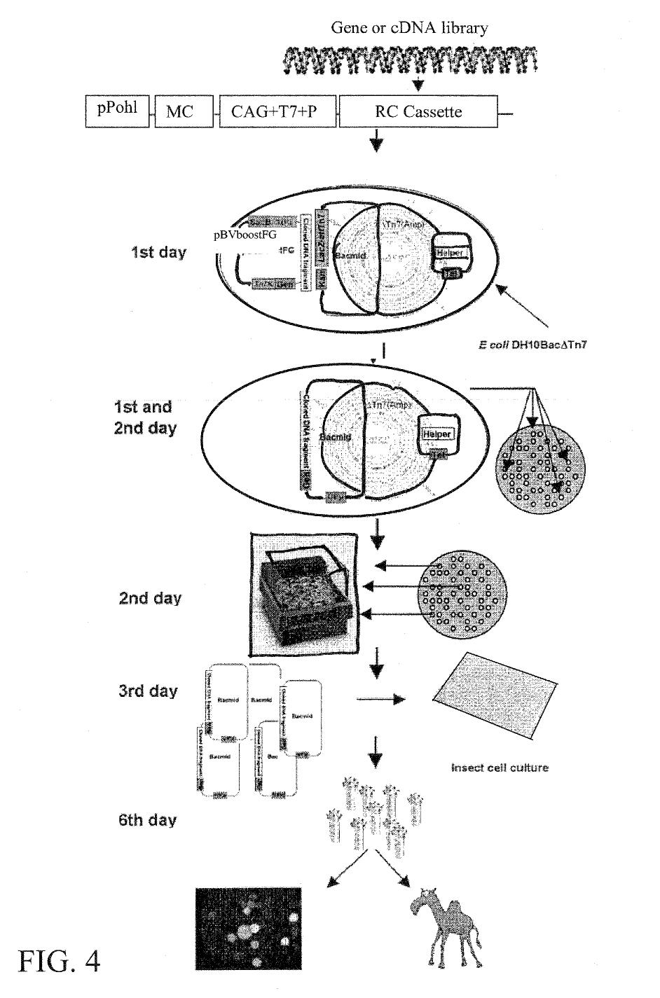

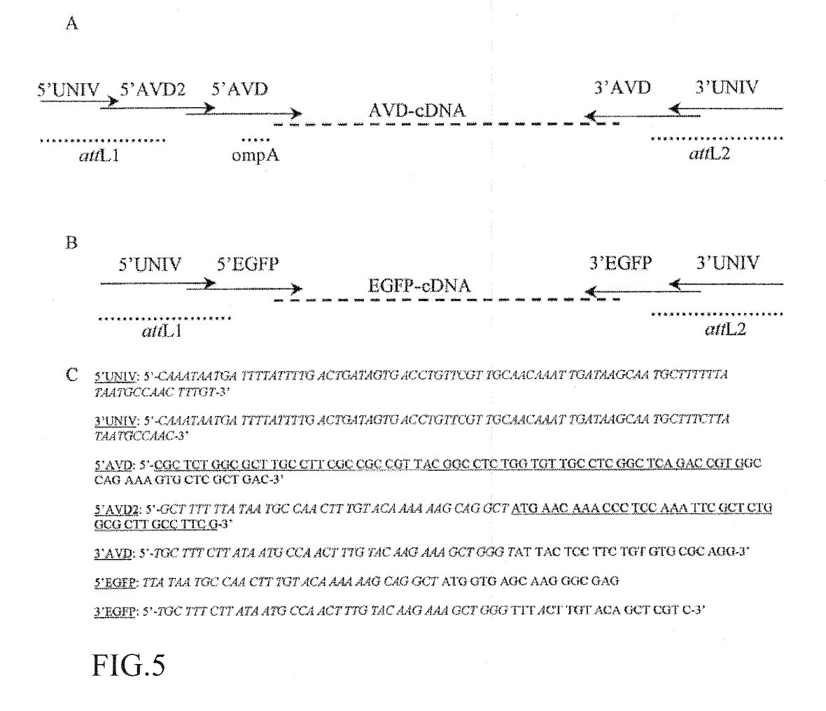

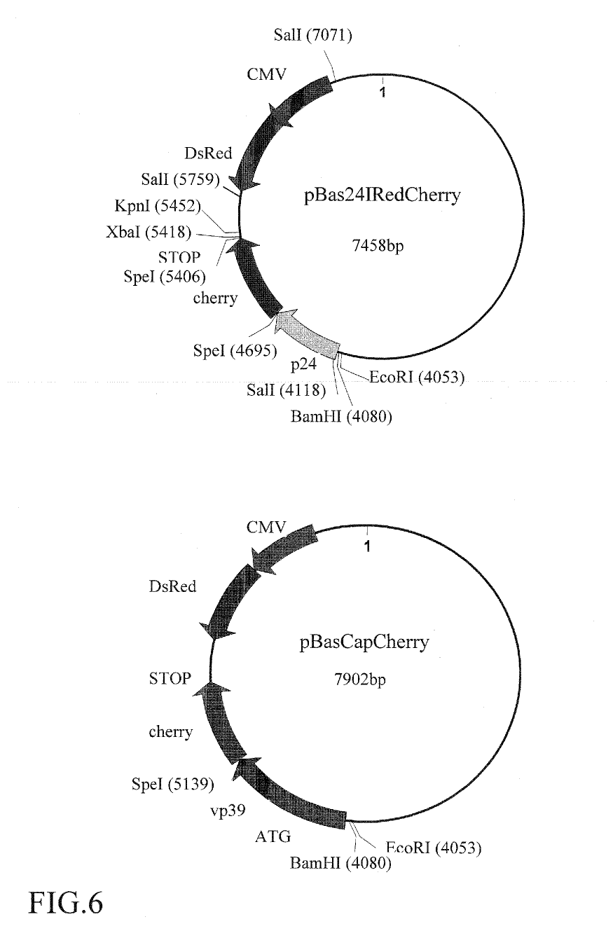

As indicated above, one aspect of the invention is a particular vector. In order to construct a vector that allow the expression of the cloned gene or cDNA library in different host systems by using only single vector without any further subcloning, four different promoters were combined in the same vector. This tetra-promoter cassette is composed of pPolh, CAG (CMVie enhancer+chicken β-actin promoter), T7lac and p10 which direct the high level expression of target genes in vertebrate cells, E. coli, and baculovirus-infected insect cells; this is described in more detail below, and shown in FIG. 3. A multiple cloning site following the pPolh promoter allows an option to modify the properties of baculoviruses or to express a marker gene to detect the synthesis of recombinant baculoviruses as described here. To allow an efficient recombinational cloning of the desired libraries (or genes/cDNAs) into the vector, the site-specific RC cassette of bacteriophage lambda containing attR1/2 sites, that makes the vector a destination vector for this recombinational cloning system, was included into plasmid. To further enable the fast and high-throughput production of recombinant baculoviruses, using the tetra-promoter-RC cassette, it may be cloned as a part of pBVboost vector that enables the zero background generation of recombinant baculoviruses, which makes it suitable for library screening. A flow chart showing how to clone and generate a desired baculoviral library in practice is shown in FIG. 4.

There are several points that make pBVboostFG-based systems a universal choice as a library screening vector. One of its main benefits is the suitability for many alternative host systems: the library (or single gene/cDNA) can be expressed in E. coli, insect cells, mammalian cells and even in intact animals in vivo by using the produced baculovirus vectors. The last option is the most important, because it provides a rapid transition from in vitro library screening to animal testing without any further subcloning steps and therefore it markedly facilitates the screening of disease-related genes. In this context, the tropism of the baculoviruses is one of the broadest of the viral gene transfer vectors studied.

A second strength of the system relies on the effective cloning scheme to generate libraries containing baculoviruses without wild-type background. It is based on two consecutive RC steps including a site-specific recombination of bacteriophage lambda and an improved mini Tn7 transposition system. The use of the RC strategy in the library construction provides several benefits over conventional restriction enzyme/ligase based cloning methods. Firstly, the lack of restriction enzyme digestions during cloning improves the fidelity of the full-length library because the aspired clones will not be digested from the internally occurring restriction sites. Secondly, the used RC system of the bacteriophage lambda provides a much better cloning efficiency than restriction-ligation based strategies. Furthermore, the site-specific recombination system of the bacteriophage lambda is reversible, in contrast to many other corresponding site-specific recombinase systems. This feature means that any fragment cloned into the novel vector can be easily transferred to any other vector utilising the same system and vice versa.

The high cloning efficiency combined with the rapid and background-free baculovirus generation yields representative libraries more facile than has been possible by homologous recombination or by conventional cloning methods. Because recombinant baculovirus genomes in this system are generated in E. coli, there is no need to carry out plaque purifications to isolate separate clones. This also facilitates screening and generation of annotated libraries.

A further advantage of using baculovirus libraries is that long DNA inserts can be screened. Also, the RC steps used in the library construction allow the transfer of long inserts. In contrast, recent adenoviral and retroviral gene transfer vectors can incorporate less than 8 kb of foreign DNA into their genomes. The construction of baculovirus libraries with pBVboostFG based system starting from extracted poly-A RNA can be accomplished within one week (FIG. 4). After screening and identification of candidate clones, virus amplification for in vivo testing can be accomplished within 1-2 weeks.

The presence of a second baculoviral promoter such as pPolh in the vector, separated from the RC schema of the bacteriophage lambda, enables the cloning of additional properties into the generated baculoviral library. This feature is exemplified by the cloning of the fluorescent marker under pPolh for the identification of the produced recombinant baculoviruses. Other, corresponding approaches are pseudotyping of the virus library or modification of the baculoviral coat or capsid by cloning gp64 or vp39 fusion proteins under the pPolh promoter, which may allow a more specific and more efficient targeting of the produced viruses into or inside specific cell types.

The following Table gives vectors used in this study.

| Vector | Description |
|---|---|
| pBVboost | Base vector for other constructs, |
| | allows high-throughput production |
| | of recombinant baculoviruses |
| | (Airenne et. al) |
| pBVboostFG | A derivative of pBVboost, |
| | compatible with recombinational |
| | cloning and universal expression |
| pBVboostFGR | A derivative of pBVboostFG, |
| | contains additional marker gene |
| | DsRed that is functional in insect |
| | cells |
| pBVboostFG + AVI | A derivative of pBVboostFG for the |
| | expression of ompA-avidin |
| pBVboostFG + EGFP | A derivative of pBVboostFG for the |
| | expression of EGFP |
| pBVboostFGR + EGFP | A derivative of pBVboostFGR for |
| | the expression of EGFP |

The following Examples illustrate the invention.

### Example 1

### Capsid Display Vector—vp39

In order to construct a general baculovirus vector for capsid display, the region corresponding to nucleotides (nt) 469-1506 of vp 39 (Genbank:M22978) was amplified from the purified bacmid DNA (Luckow et al, J. Virol. 67, 4566-4579, 1993) by polymerase chain reaction (PCR). The forward primer was 5′-TT GAA AGA TCT GAA TTC ATG CAC CAC CAT CAC CAT CAC GGA TCC GGC GGC GGC GGC TCG GCG GCT AGT GCC CGT GGG T -3′ (specific sequence for nt 469-486 of vp39 gene in bold; BglII, EcoRI, BamHI, sites underlined; 6× Histidine tag with start codon in italics); the reverse primer was 5′-TT CTG GGT ACC GCt tta ATG GTG ATG ATG GTG GTG TCT AGA GCt tta ACT AGT GAC GGC TAT TCC TCC ACC -3′ (specific sequence for nt 1489-1506 of vp39 gene in bold; KpnI, XbaI and SpeI sites underlined; 6× Histidine tag in italics; stop codon in small caps). PCR was performed essentially as described by Airenne et al, Gene 144:75-80, 1994, except annealing was set to 58° C. Amplified fragment was digested with BglII and KpnI enzymes and purified as described in Airenne et al, supra. The purified PCR product was cloned into BamHI+KpnI-digested pFastBAC1 vector (Invitrogen, Carlsbad, USA). The resulted plasmid was named as pBACcap-1. The nucleotide sequence was confirmed by sequencing (ALF; Amersham Pharmacia Biotech, Uppsala, Sweden).

### Preparation of EGFP-Displaying Viruses

cDNA encoding EGFP (enhanced green fluorescent protein) was amplified from the pEGFP-N1 plasmid (Genbank:U55762, Clontech, Palo Alto, USA) by PCR and cloned into the pBACcap-1. Two sets of primers were used to enable EGFP fusion both to N- and C-terminal ends of the vp39. For the N-terminal fusion, the forward primer was 5′-CGG GAT GAA TTC GTC GCC ACC ATG GTG AGC AAG GGC GAG GAG -3′ (specific sequence for nt 670-699 of pEGFP-N1 in bold; EcoRI site in italics), and the reverse primer 5′-GCG GCC GGA TCC CTT GTA CAG CTC GTC CAT GCC-3′ (specific sequence for nt 1375-1395 of pEGFP-N1 in bold; BamHI site in italics). The amplified fragment which corresponded to nt 670-1395 of pEGFP-N1 was cloned into EcoRI/BamHI site of the SpeI/XbaI-deleted pBACcap-1. The resulting plasmid was named pEGFPvp39.

For the C-terminal version, the forward primer was 5′-GTC GCC ACT AGT GTG AGC AAG GGC GAG GAG CTG -3′ (specific sequence for nt 682-702 of pEGFP-N1 in bold; SpeI site in italics), and the reverse primer 5′-AGA GTC ACT AGT GCt tta CTT GTA CAG CTC GTC CAT GCC -3′ (specific sequence for nt 1375-1398 of pEGFP-N1 in bold; SpeI site in italics; stop codon in small caps). The amplified fragment which corresponded to nt 682-1398 of pEGFP-N1 was cloned into SpeI site of the pBACcap-1. The resulting plasmid was named pvp39EGFP. The nucleotide sequences were confirmed by sequencing (ALF).

Recombinant viruses were generated using the Bac-To-Bac System™ according to manufacturer's instructions (Invitrogen). Viruses were concentrated and gradient-purified, as described by Airenne et al, Gene Ther. 7:1499-1504, 2000. Virus titer was determined by end-point dilution assay on Sf9 cells. Sterility tests were performed for virus preparations and they were analysed to be free of lipopolysaccharide and mycoplasma contamination.

### Immunoblotting

Samples corresponding to about 60,000 infected cells or virus from 4 ml of culture medium were loaded onto 10% SDS-PAGE under reducing conditions. The gel was blotted onto nitrocellulose filter and immunostained as described by Airenne et al (1994), supra. Polyclonal rabbit anti-EGFP (Molecular Probes Inc., Eugene, USA) was used as a primary:antibody (1:4000) and goat anti-rabbit serum as a secondary antibody (1:2000) (Bio-Rad, Hercules, USA). Molecular weight standard in the SDS-PAGE was from Bio-Rad.

### Electron Microscopy

For immunoelectron microscopy, vp39EGFP baculovirus particles were bound to formwar-coated metal grids treated with 5% foetal calf serum in PBS, allowed to react with anti-GFP antibody (1:600 dilution, 30 min), and washed with PBS. Grids were then treated with gold-conjugated protein A for 25 min (5 nm in diameter, G. Posthuma and J. Slot, Utrecht, The Netherlands) and washed with PBS for 25 min. The grid was fixed with 2.5% glutaraldehyde and contrasted and embedded using 0.3% uranyl acetate in 1.5% methyl cellulose. The human hepatoma cell line HepG2 and human endothelial aortic hybridoma cells (EAHy926, Dr. Edgell, Univ. N. Carolina, USA) transduced with the virus were fixed with 2.5% glutaraldehyde for 1 h at room temperature and then with 1% osmium tetroxide for 1 h at +4° C. After dehydration, cells were stained with 2% uranyl acetate for 30 min at room temperature, embedded in Epon and sectioned for electron microscopy. Sections were stained with lead citrate and uranyl acetate. Samples were examined using a JEM-1200 EX electron microscope (Jeol Ltd., Tokyo, Japan).

### Immunofluorescence and Confocal Microscopy

Subconfluent EAHY, HepG2, MG63 (human osteosarcoma) and NHO (normal human osteoblast) cell cultures were infected by vp39EGFP baculovirus as follows: cells were first washed with PBS on ice, the virus was added in DMEM containing 1% foetal calf serum using a multiplicity of transductions of 80-100 pfu per cell, and incubated for 1 h on ice (rocking). The effect of lysosomal pH on baculovirus entry was tested by incubating the cells in the medium supplemented with monensin at 0.5 μM. Cells were washed with PBS containing 0.5% BSA. Then, DMEM (containing 10% serum) was added and cells were incubated for various time periods at 37° C. and finally fixed with 4% paraformaldehyde in PBS for 20 min. Cells were labelled with EEA1 (early endosome antigen 1; BD Transduction Laboratories, Lexington, Ky.). Goat secondary antibodies against mouse antibodies (Alexa red 546 nm; Molecular Probes Inc., Eugene, Oreg.) were used in the labelling. The cells were mounted in mowiol and examined with an Axiovert 100 M SP epifluorescence microscope (Carl Zeiss, Jena, Germany) and a confocal microscope (Zeiss LSM510). For visualising EGFP and Alexa red 546, multitracking for 488 and 546 laser lines was used in order to avoid false co-localisation. Live confocal microscopy on HepG2 and EAHY cells was performed as follows: cells were plated on chambered coverglasses (Nalge NUNC, Naperville, Ill.). After virus binding on ice, cells were transferred to the confocal microscope with a heated working stage and objective controlled by Tempcontrol 37-2 (Carl Zeiss, Jena, Germany). Cells that were positive for EGFP were scanned with various time intervals using the programme in LSM 510 software (program version 2.3; Carl Zeiss, Jena, Germany).

In Vivo Injection into Rat Brain

Male Wistar rats (320-350 g) were anaesthetised intraperitoneally with a solution (0.150 ml/100 g) containing fentanyl-fluanisone (Janssen-Cilag, Hypnorm®, Buckinghamshire, UK) and midazolame (Roche, Dormicum®, Espoo, Finland) and placed into a stereotaxic apparatus (Kopf Instruments). A burr hole was done into the following stereotaxic coordinates: 1 mm to the satua sagittalis and +1 mm to bregma. 100 [symbol] λ of the EGFPvp39 or vp39EGDP baculoviruses (0.9×10⁻¹⁰ pfu/ml) in 0.9 N NaCl was injected during 4×5 min periods using a Hamilton syringe with a 27-gauge needle to a depth of 3.5 mm. Animals were sacrificed with CO₂, 7 h after the gene transfer. Rats were perfused with PBS by the transcardiac route for 10 min, followed by fixation with 4% paraformaldehyde/0.15 M sodium-phosphate buffer (pH 7.4) for 10 min. Brains were removed, and snap-frozen with isopenthane, and 40 μm thick frozen sections were prepared. Slides were immediately analysed with fluorescence microscopy (Olympus AX70 microscope, Olympus Optical, Japan) and data were collected with Image-Pro Plus software.

### Characterisation of EGFP-Displaying Viruses

Sf9 cells infected with EGFPvp39 or vp39EGFP-encoding viruses produced the expected 67 kDa bands in immunoblots. The same results were obtained from the gradient-purified virus preparations. The results suggested that both vp39 variants were efficiently produced in insect cells and incorporated into virus particles. However, to confirm that the fusion proteins were part of the virus capsids, the vp39EGFP virus was gradient-purified and incubated with anti-EGFP, labelled with protein A gold, and analysed by electron microscopy. The viral capsids showed a typical rod-shaped morphology, and the surfaces of the unenveloped capsids were heavily gold-labelled. Intact virions were not labelled. Thus, a large quantity of EGFP was evenly distributed around the recombinant baculovirus capsid.

In order to estimate the amount of the incorporated EGFP per virus particle, serial dilutions of the purified virus particles were immunoblotted and compared to the known amount of the purified EGFP. Analysis indicated that about 860 EGFP molecules were incorporated per virus particle. 590 EGFP molecules per capsid were measured by comparing the detected fluorescence of the vp39EGFP virus preparation to EGFP control. The high incorporation rate was also supported by Coomassie-stained SDS-PAGE, according to which a high proportion of the capsid was made of the vp39EGFP. Assembly of the viruses was not affected by the fusion protein, since the titers of the gradient-purified and concentrated (200×) EGFPvp39 and vp39EGFP viruses were 9.5×10⁹ and 8.8×10⁹ pfu/ml, respectively.

### Baculovirus-Mediated Transduction

The intracellular route of vp39EGFP virus was followed by monitoring EGFP-tagged capsids and fluorescently labelled cellular compartments by confocal microscopy. EAHY, HepG2, MG63 and NHO cells were transduced for various time periods and the co-localisation of the virus with an early endosome antigen 1 (EEA1) was studied. EAHY, MG63 and NHO cells were chosen since it has been found that they are completely non-permissive for baculovirus transduction with LacZ-baculovirus. No blue-stained cells were detected in the plates even in the presence of 10 mM sodium sodium butyrate (which enhances gene expression) by X-gal staining with a very high multiplicity of transduction (1000) while the amount of blue-stained rabbit aortic smooth muscle cells (RAASMC) were in agreement with results presented by Airenne et al (2000), supra.

Baculovirus is known to enter cells via the endocytic pathway. Before the capsid is delivered to the nucleus, the baculovirus envelope fuses with the membrane of the early endosome under mildly acidic conditions with the help of the viral gp64. After 30 min post-transduction (p.t.), it could be seen that the virus was still present in early endosomes in both HepG2 and EAHY cells. 4 and 24 h p.t. the virus did not colocalise with the EEA1 in the EAHY cells, suggesting that it had already escaped from the early endosomes. However, in these cells, the capsids did not enter the nuclei, whereas in HepG2 cells the capsids were seen in the nuclei as bright spots 4 h p.t. In EAHY cells the number of capsid (EGFP) positive nuclei was very low (0.1%) whereas almost all nuclei were positive in HepG2 cells 4 h p.t. (91%). At 24 h p.t., EGFP was no longer clearly distinguished in HepG2 cell nuclei, suggesting that the capsids had disassembled, whereas they were still present in the cytoplasm in EAHY cells. Fluorescent labelling of recycling early endosomes with rab11 and late endosomes and lysosomes with anti-CD63 showed no colocalisation with EGFP at 24 h p.t. in EAHY cells, suggesting that the virus capsid was not in the endocytic pathway. Electron microscopy of EAHY cells at 4 h p.t. confirmed that the virus capsids were free in the cytoplasm, further suggesting that they had escaped from the early endosomes. In HepG2 cells, the capsids were present in the nuclei at 4 h p.t., showing that intact capsids were transported into the nucleus after release from the early endosomes. Live imaging of vp39EGFP virus supported the results of colocalisation studies. Electron microscopy of EAHY cells confirmed that no virus capsids were present in the nuclei at 4 h p.t. In order to find out whether the block in the nuclear entry of baculovirus in the EAHY cells is also valid for other non-permissive cells, MG63 (FIG. 4) and NHO cells were studied by vp39EGFP virus. The results suggest a general block in the nuclear entry of baculovirus capsid in the non-permissive cells.

Transduction of the cells in the presence of monensin led to a block in the virus capsid entrance into the cytoplasm. Monensin inhibits early endosome acidification and causes accumulation of the cargo in the early endosomes. In HepG2 and EAHY cells, monensin caused accumulation of the virus in EEA1 positive early endosomes at 4 h p.t. The results thus suggest that the virus follows the same pathway in permissive and non-permissive cells. In both cell types baculovirus is taken up by adsorptive endocytosis, followed by a pH-dependent fusion of the envelope with endosome as has previously been shown to occur in insect and mammalian cells.

### Visualisation of Virus in Rat Brain In Vivo

In order to investigate the utility of vp39EGFP for baculovirus biodistribution studies, an aliquot of the virus was injected into the rat brain. The virus was still clearly seen at 7 h after injections into the right corpus callosum of rat brain near the injection site. Thus, the vp39EGFP baculovirus can be used for more detailed biodistribution studies in vivo.

### Example 2

### Bacterial Strains, Plasmids, Cell Lines and Viral DNA

E. coli strain DH5α (Invitrogen, USA) was used for propagation of plasmids. DH10Bac cells and pFastbac1 were obtained from Invitrogen. pDNR-LIB vector containing SacB gene was purchased from BD Biosciences Clontech, USA.

### Construction of Modified Donor Vector

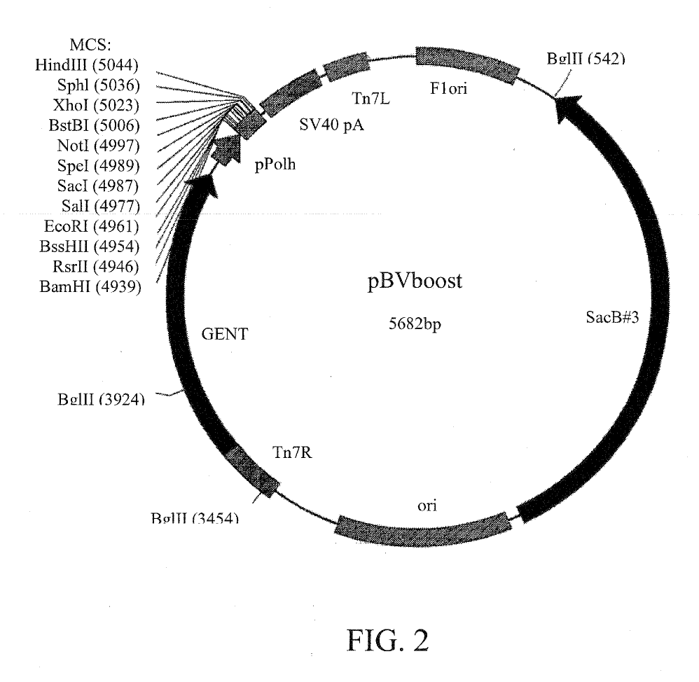

The modified donor vector was constructed by replacing the Ampicillin resistance gene in pFastbac1 vector with Bacillus subtilis levansucrase gene (SacB) from pDNR-LIB vector. In practice, pFastbac1 vector was cut by BspHI restriction enzyme, and the linear vector backbone was purified by gel electrophoresis. The SacB expression cassette was obtained from pDNR-LIB by polymerase chain reaction (PCR) with the primers DNR5′: 5′-GTTATTCATGAGATCTGTCAATGCCAATAGGATATC-3′ (sequence for nt 1263-1282 of pDNR-LIB in bold; BspHI and BglII sites underlined), DNR3′: 5′-TTAGGTCATGAACATATACCTGCCGTTCACT-3′ (sequence for nt 3149-3179 of pDNR-LIB in bold; BspHI site underlined). PCR was performed essentially as described by Airenne et al (1994), supra, except that annealing was carried out at 58° C. and EXT DNA polymerase (Finnzymes, Helsinki, Finland) was used for amplification. The amplified fragment was digested with BspHI and purified as described in Airenne et al, (1994), supra. The purified PCR product was cloned into a BspHI-digested pFastbac1 vector (Invitrogen, Carlsbad, USA) for orientation shown in FIG. 2. The resulting plasmid was named pBVboost. The SacB#3 cassette nucleotide sequence was confirmed by DNA sequencing (ALF; Amersham Pharmacia Biotech, Uppsala, Sweden).

Construction of Chromosomal attTn7 Blocked E. coli Strain

In order to block the cryptic attTn7 site in DH10Bac, pBVboost was cut by BseRI/AvrII. The excised gentamycin resistance was substituted by ampicillin resistance cassette (ARC) from pFastbac1. The ARC was obtained by PCR with the primers DH10Bacinttn7destroybyamp5′: 5′-AAATATGAGGAGTTACAATTGCTAATTAATTAATTCGGGGAAATGTGCGCGGAA -3′ (sequence for nt 471-490 of pFastbac1 in bold; BseRI site underlined), DH10Bacinttn7destroybyamp3′: 5′-CTTGGTCCTAGGATTACCAATGCTTAATCAGTG -3′ (sequence for nt 1430-1449 of pFastbac1 in bold; AvrII site underlined). The PCR was performed as described above. The amplified fragment was digested with BseRI/AvrII and purified as above. The purified PCR product was cloned into a BseRI/AvrII-digested pBVboost. The resulting plasmid was named pBVboostΔamp. The nucleotide sequence of Ampicillin cassette was confirmed by DNA sequencing (ALF; Amersham Pharmacia Biotech, Uppsala, Sweden).

DH10Bac cells were transformed by pBVboostΔamp. Single blue colonies were picked from LB-plates containing 50 μg/ml kanamycin sulphate (Kan), 10 μg/ml tetracycline (Tet), 50 μg/ml ampicillin (Amp), 50 μg/ml X-gal, 1 mM IPTG and 10% sucrose in 5 ml LB-medium. Next day colonies were screened for the presence of intact Bacmids by PCR as described by Donahue, Focus 17, 101-102, 1995. Colonies resulting in 325 bp bands (sign of intact Bacmid) in gel electrophoresis were further studied for the absence of donor plasmid by running samples of purified plasmid DNA (Wizard minipreps; Promega. Madison, USA) in gel. Resulting clones were preserved in −70° C. as E. coli DH10BacΔTn7.

### Preparation of Electro-Competent Cells

In order to prepare electro-competent cells, single colonies from LB-plates (Kan, Tet for DH10Bac or Kan, Tet and Amp for DH10Bac_Tn7 cells at above concentrations) were inoculated into 10 ml of Super broth (SB; 30 g Tryptone, 20 g Yeast Extract, 10 g 3-N-morpholinopropanesulfonic acid, 1 l water, pH 7.0) with appropriate antibiotics. Suspensions were cultivated overnight at 37° C. on a shaker. One liter of SB with 5 ml of 2 M glucose was then inoculated with 5 ml of overnight culture until the optical density of the new culture reached 0.8-0.9 (about 2-4 hours) at 600 nm. Culture was then chilled on ice for 15 min and centrifuged at 1500 g for 15 min at 4° C. Cells were washed with 800, 500, 300, 200 and 100 ml of ice-cold water/10% glycerol and centrifuged as above. Finally cells were suspended in a total volume of 3-4 ml of 10% glycerol and preserved in 40 μl aliquots at −70° C.

Transposition into Bacmids and Production of Recombinant Baculoviruses

Transposition was performed by electro-transforming 40 μl of DH10Bac or DH10BacΔTn7 with pFastbac1 or pBVboost donor vector. Electro-transformation was performed as described by Gibco BRL, using BIO-RAD Gene Pulser II system (Hercules, USA). The cells were allowed to recover 4 h post transformation at 37° C. with vigorous shaking. The cultures were plated on LB-plates supplemented with 7 μg/ml gentamycin (Gent) and Tet (10 μg/ml) with and without 10% sucrose. Colonies were studied for the presence of recombinant baculovirus genomes by PCR as described above. The recombinant viruses were generated according to the protocol provided by the Bac-To-Bac system (Invitrogen).

### Results

The transposition efficacy in the DH10Bac or DH10BacΔTn7 (in which the chromosomal attTn7 site is occupied) cells was studied using the original pFastbac1 or pBVboost donor vectors and the results were compared. As expected, the use of pBVboost resulted in a significant increase in the efficacy of the generation of recombinant bacmids in the presence of sucrose. Over ten-fold increase in the transposition efficacy (white colonies) was detected in favor of pBVboost in DH10Bac cells. Furthermore, the transformation of DH10BacΔTn7 with pBVboost resulted typically in 100% white colonies as compared to only 27% in the pFastbac1 plates. The presence of recombinant bacmids in the morphologically white colonies was proved by PCR. Notably, the use of DH10BacΔTn7 strain also yielded a significant increase in the recombinant bacmids with pFastbac1.

### Example 3

### Construction of pBVboostFG and pBVboostFGR

In order to allow recombinational cloning into planned vector, the Gateway cloning cassette A (Invitrogen) were inserted into modified pTriEx-1.1 vector (Novagen). The constructed cassette was cloned into the pBVboost vector that enables rapid generation of baculoviruses (Example 2) and the resultant vector was designated as pBVboostFG (FIG. 3). To construct a marker gene-containing version of pBVboostFG, the DsRed encoding sequence (from pDsRed2-N1 vector, Clonetech) was subcloned into MCS of the pBVboostFG under a polyhedron promoter (pPolh). This vector was named pBVboostFGR.

Cloning of Avidin and EGFP into pBVboostFG and pBVboostFGR Vectors

The DNA-construct containing bacterial ompA secretion signal fused to avidin cDNA flanked with attL1 (5′) and attL2 (3′) sites required for recombinational cloning was obtained using SES-PCR in three steps (FIG. 5). This product was LR-cloned (Invitrogen) into pBVboostFG and the resultant plasmid was named pBVboostFG+avi. The EGFP-construct (pEGFP-N1, Clontech, Palo Alto, USA) was prepared with an identical SES-PCR procedure in two steps, after which it was cloned into pBVboostFG and pBVboostFGR. The resultant plasmids were designed as pBVboostFG+EGFP and pBVboostFGR+EGFP, respectively.

### Expression of Genes and Characterisation of Proteins

Bacterial expressions of ompA-avidin and EGFP were carried out in E. coli BL21 strain expressing T7 polymerase. For the expression of ompA-avidin, the cells were first cultured at 37° C. in the shaking culture conditions until the optical density reached 0.2 (A₅₉₅), after which the protein production was switched on by adding IPTG to the final concentration of 0.4 mM. Avidin synthesis was allowed to continue over night at room temperature. The cells were fractioned into total, periplasmic and insoluble fractions, and these fractions were subjected to 15% SDS-PAGE and transferred onto nylon bead filters. The proteins were detected by polyclonal rabbit anti-avidin antibody (1:5000), and Goat Anti-Rabbit IgG-AP (1:2000) was used as a secondary antibody. EGFP expression was carried out by growing bacteria on LB plates containing 0.4 mM IPTG and gentamycin, and the produced EGFP was detected directly from cultures under UV-light.

Recombinant baculoviruses were constructed using vectors pBVboostFG+EGFP and pBVboostFGR+EGFP as described above (Example 2). Baculoviral infections were performed in Sf9 cells (1×10⁶ cells in each well of 6-well plates) for 3 days.

To test the constructed expression cassette in mammalian cells, HepG2 and CHO were used as a test cell lines for expressing EGFP through CAG promoter. The functionality of the cassette was tested both by the baculoviral transduction and by transfection (FUGENE™ 6, Roche) using pBVboostFG+EGFP. In both tests, 150,000 cells were plated into wells of 6-well plates and, after 24 h, the cells were either transfected by 1-2 μg of plasmid DNA or transducted by virus with the MOI 300. Cells were incubated for another 24 h and imaged by fluorescence microscope.

Cloning Test Genes into pBVboostFG and pBVboostFGR

The bacterial ompA secretion signal was fused to avidin gene in order to transport the synthesised the avidin to periplasmic space of E. coli. In order to RC clone ompA-avidin and EGFP into pBVboostFG(R) in one step (FIG. 5), the long attL sites required for the cloning system were included by using SES-PCR; see Majumer et al, Gene 110, 89-94, 1992.

### Expression of Test Genes Avidin and EGFP

The expression of avidin (pBVboostFG+AVI) was efficient in BL21 E. coli and a remarkable proportion of total cellular protein was composed of avidin after over night induction. Part of the avidin was produced as insoluble inclusion bodies. The inclusion bodies as well as the total cell sample contained also a non-processed form of the protein (i.e. protein that still contained the signal peptide). In contrast, the ompA signal was cleaved off from virtually all periplasmic avidins. The functionality of periplasmic avidin was studied by binding it to biotin agarose and the whole fraction bound to agarose. The EGFP was also produced successfully as a functional form in E. coli transformed with the plasmid pBVboostFG+EGFP since it was easily detected directly from bacterial cultures growing onto LB plates.

Baculoviruses encoding EGFP were used to infect Sf9 cells. After 3 days infection, the cells were studied in fluorescent microscope. In practice, all cells were infected. Correspondingly, viruses that contained both the DsRed and EGFP infected Sf9 cells similarly.

HepG2 and CHO cells were used to show that the tetra-promoter construct works also in mammalian cells. In this case, the same EGFP construct was used as with Sf9 cells. The construct was both transducted as baculoviruses into HepG2 and CHO cells and transfected as a plasmid (pBVboostFG+EGFP) into CHO cells.

### Example 4

### Cloning of p24 and vp39 Capsid Protein Fusions

The gene encoding the capsid protein p24 was amplified from baculovirus genome by PCR using a forward primer 5′ GC TGT GGA TCC GGC GGC GGC GGC TCG AAC ACG GAC GCT CAG TCG 3′ and a reverse primer 5′CC TTA ACT AGT TTT ATT CAG GCA CAT TAA ATC 3′. The primers contained the restriction sites for BamHI and SpeI restriction enzymes (sites in bold) for cloning to the modified pBACcap-1 vector containing a cassette expressing DsRed under CMV-IE promoter. In the forward primer also a linker sequence (in italics) was included to separate the fusion partner from the p24 capsid protein. The vp39 gene was removed from the vector by digesting with BamHI and SpeI and the p24 fragment was cloned to the vector after digestion with the same enzymes. The resulting plasmid was named pBac24IRed. Fluorescent proteins tdTomato and mCherry were cloned as C-terminal fusions to vp39 and p24 capsid proteins. Sequences were amplified from pRSET-B mCherry and pRSET-B tdTomato plasmids by PCR with primers containing restriction sites for SpeI (in bold). The sequence of the forward primer was 5′ AA GGA ACT AGT GTG AGC AAG GGC GAG GAG 3′ and the reverse primer 5′ TC GAA ACT AGT CTT GTA CAG CTC GTC CAT 3′. The vector constructs were verified by sequencing (AIVIN laite) and recombinant baculoviruses were produced with BVboost system (Airenne et al. 2003, Laitinen et al. 2005). Viruses were named Vp39Tomato, Vp39Cherry and p24Cherry.

### Generation and Characterization of Recombinant Viruses.

Both tdTomato and mCherry fluorescent proteins were successfully fused to the capsid protein vp39 (plasmid maps in FIG. 6.) and high-titer viruses were generated. Concentrated and purified viruses were observed as bright fluorescent particles in transduced cells. The fusion to the p24 capsid protein was achieved with the monomeric mCherry protein.

1. A baculovirus of which the capsid has been modified to display one or more heterologous peptides.

2. The baculovirus according to claim 1, wherein vp39, p24 or p80 is modified.

3. The baculovirus according to claim 2, wherein vp39 is modified.

4. The baculovirus according to claim 3, wherein vp39 is modified with a fusion protein at the N- and/or C-terminus.

5. The baculovirus according to claim 1, wherein the modification allows nuclear or subcellular targeting.

6. The baculovirus of which the genome has been modified to express one or more heterologous peptides in its capsid, as defined in claim 1.

7. The baculovirus according to claim 6, wherein the baculovirus contains at least 3 heterologous genes.

8. The baculovirus according to claim 6, wherein one or more heterologous genes are at least 10 kb long.

9. The baculovirus according to claim 6, wherein the heterologous peptides are encoded by human genes.

10. A method for delivering a peptide into the nucleus of a cell wherein said method comprises the use of a baculovirus according to claim 1.

11. The method according to claim 10, wherein the cell is an insect cell.

12. The method according to claim 10, wherein the cell is a mammalian cell.

13. A method for selecting a target gene, which comprises the steps of:
(i) generating a library of genes or genomic fragments cloned in a baculovirus according to claim 1;
(ii) transforming a host cell with the baculovirus; and
(iii) detecting gene expression under predetermined conditions.

14. The method according to claim 13, wherein the predetermined conditions comprise a set of different conditions under which expression of the target gene may or may not be detected.

15. The method according to claim 14, wherein the different conditions comprise limiting dilution.

16. The method according to claim 13, wherein step (iii) comprises identification of a phenotype.

17. The method according to claim 13, wherein step (iii) is repeated following selection of one or some of the products of the predetermined conditions.

18. The method according to claim 13, which additionally comprises characterising the gene expressed under the predetermined conditions.

19. A method for generating recombinant baculoviruses, comprising:
(i) incorporating a lethal gene into a donor plasmid comprising an expression cassette;
(ii) transposing the expression cassette from the donor plasmid into a bacmid in E. coli cells to form a recombinant bacmid, wherein the lethal gene product kills the cells still harbouring the donor vector;
(iii) extracting the recombinant bacmids; and
(iv) transfecting insect cells with recombinant bacmids to form recombinant baculoviruses.

20. The method according to claim 19, wherein the transposition is TN7-mediated

21. The method according to claim 19, wherein the lethal gene is a mutated levansucrase gene.

22. The method according to claim 18, wherein the chromosomal attTn7 site in the E. coli strain is occupied.

23. The method according to claim 22, wherein the E. coli strain is DH10BacΔTn7.

24. The method according to claim 19, wherein the expression cassette contains/is driven by a universal promoter.

25. The method according to claim 24, wherein the universal promoter is a tetra-promoter composed of a CMVie enhancer and chicken β-actin promoter (CAG), Y7lac, pPolh and p10.
